(19) United States
(12) Plant Patent Application Publication
(10) Pub. No.: US 2006/0137054 P1
(43) Pub. Date: Jun. 22, 2006

(54) SCABIOSA PLANT NAMED 'BALHARBU'

(50) SCABIOSA HYBRIDA; SCABIOSA

(75) Inventor: Scott C. Trees, Shell Beach, CA (US)

Correspondence Address: WOOD, PHILLIPS, KATZ, CLARK & MORTIMER, 500 W. MADISON STREET, SUITE 3800, CHICAGO, IL, 60661, US

(73) Assignee: Ball Horticultural Company

(21) Appl. No.: 11/015,231

(22) Filed: Dec. 17, 2004

(51) Int. Cl. A01H 5/00

(52) U.S. Cl. PLT/263

(57) ABSTRACT

A new and distinct cultivar of Scabiosa plant named ‘Balharbu’ characterized by its large inflorescence form, pale lavender-colored flowers, freely branching character, dark green-colored foliage, and vigorous, upright growth habit.

### BACKGROUND OF THE INVENTION

The present invention relates to a new and distinct cultivar of Scabiosa plant botanically known as Scabiosa hybrida, and hereinafter referred to by the cultivar name ‘Balharbu’.

The new Scabiosa originated in a controlled breeding program in Arroyo Grande, Calif. during October 1999. The objective of the breeding program was the development of Scabiosa cultivars that have a vigorous upright growth habit and large flowers.

The female parent of the new cultivar was the Scabiosa columbaria cultivar Butterfly Blue, not patented, characterized by its small flowers and compact growth habit. The male parent of the new cultivar was the Scabiosa anthemifolia cultivar ‘Giant Blue’, U.S. Plant Pat. No. 12,167, characterized by its large flowers and whole-leaf foliage. Seed from the above stated cross-pollination was germinated and grown to maturity. One plant within the progeny was discovered and selected by the inventor during October 2000 in a controlled environment at Arroyo Grande, Calif.

Asexual reproduction of the new cultivar by terminal stem cuttings since October 2000 at West Chicago, Ill. has demonstrated that the new cultivar reproduces true to type, with all the characteristics, as herein described, firmly fixed and retained through successive generations of such asexual propagation.

### SUMMARY OF THE INVENTION

The following characteristics of the new cultivar have been repeatedly observed and can be used to distinguish ‘Balharbu’as a new and distinct cultivar of Scabiosa plant:

1. Large inflorescence form.

2. Pale lavender-colored flowers.

3. Dark green-colored foliage.

4. Freely branching character.

5. Vigorous, upright growth habit.

Plants of the new cultivar differ from plants of the female parent primarily in flower size and plant vigor and from plants of the male parent primarily in leaf shape.

Plants of the new cultivar are most similar to the cultivar Butterfly Blue, not patented. However, in side-by-side comparisons, plants of the new cultivar differed from plants of ‘Butterfly Blue’ in the following characteristics:

1. Plants of the new cultivar exhibit a more vigorous growth habit than do plants of ‘Butterfly Blue’.

2. Plants of the new cultivar form larger inflorescences than do plants of ‘Butterfly Blue’.

### BRIEF DESCRIPTION OF THE PHOTOGRAPHS

The accompanying photographs show, as nearly true as it is reasonably possible to make the same in color illustrations of this type, typical flower and foliage characteristics of the new cultivar. Colors in the photographs differ slightly from the color values cited in the detailed description, which more accurately describe the colors of the new cultivar. The plants were grown in 10 cm pots for 8 weeks in a greenhouse at West Chicago, Ill.

FIG. 1 illustrates a side view of the overall growth and flowering habit of the new cultivar with one plant per pot.

FIG. 2 illustrates a close-up view of an individual flower of the new cultivar.

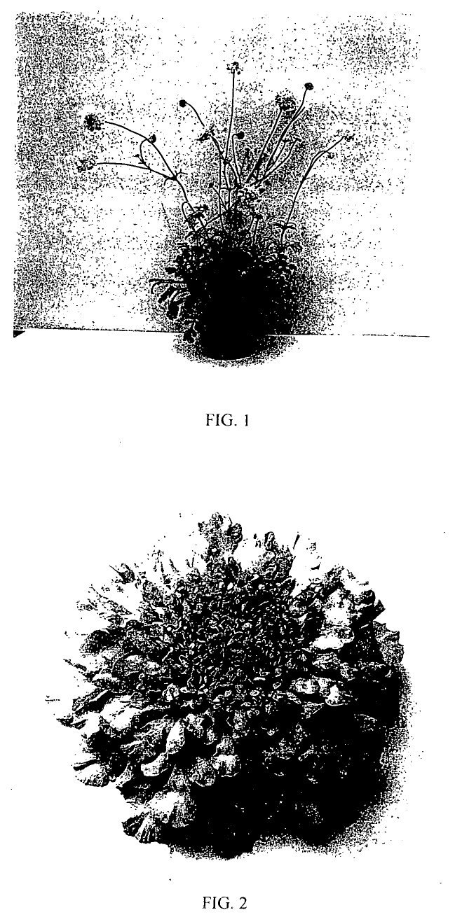

### DETAILED BOTANICAL DESCRIPTION

The new cultivar has not been observed under all possible environmental conditions to date. Accordingly, it is possible that the phenotype may vary somewhat with variations in the environment, such as temperature, light intensity, and day length without, however, any variance in genotype.

The chart used in the identification of colors described herein is the R.H.S. Colour Chart of The Royal Horticultural Society, London, England, 2001 edition, except where color terms of ordinary significance are used. The color values were determined on May 14, 2004 between 1:00 and 3:00 p.m. under natural light conditions.

The following descriptions and measurements describe plants produced from cuttings taken from stock plants and grown in a double polycarbonate-covered greenhouse under conditions comparable to those used in commercial practice. The plants were grown at West Chicago, Ill. for 8 weeks in 10 cm pots while utilizing a soilless growth medium. Greenhouse temperatures were maintained at approximately 65°-75° F. (18°-24° C.) during the day and approximately 50°-58° F. (10°-14° C.) during the night. Greenhouse light levels were maintained at 5,000 to 9,000 footcandles during the day.
Botanical classification: Scabiosa hybrida cultivar Balharbu.
Parentage:
   Female parent.—Scabiosa columbaria cultivar Butterfly Blue, not patented.
   Male parent.—Scabiosa anthemifolia cultivar Giant Blue, U.S. Plant Pat. No. 12,167.
Propagation:
   Type cutting.—Terminal stem.
   Time to initiate roots.—Approximately 9 to 12 days.
   Time to produce a rooted cutting.—Approximately 28 to 35 days.
   Root description.—Fine, fibrous.
   Rooting habit.—Freely branching.
Plant description:
   Crop time.—Approximately 5-6 weeks from a rooted cutting in a 10 cm pot.
   Growth habit.—Vigorous, with each lateral branch forming a single flower head.
   General appearance and form.—Upright.
   Size.—Height from soil level to top of plant plane: Approximately 55 cm. Height of foliage only: Approximately 12.9 cm. Plant width (area of spread): Approximately 27.5 cm.
   Branch.—Quantity of flowering branches per plant at any one time: Approximately 4. Shape: Round in cross section. Strength: Strong. Length: Approximately 34.4 cm. Diameter: Approximately 4.5 mm. Internode length at middle of branch: Approximately 4.7 mm. Texture: Strigose. Color: 138A.
   Basal foliage.—Quantity of leaves per branch: Approximately 8. Type: Simple. Fragrance: None. Arrangement: Opposite. Aspect: Almost at a right angle to the stem. Shape: Spatulate. Apex: Cuspidate. Base: Truncate. Margin: Coarsely lobed. Venation pattern: Pinnate. Length: Approximately 8.1 cm. Width: Approximately 6.1 cm. Texture: Upper and lower surfaces are villous. Color of mature foliage: Upper surface: 147A with venation of 145C. Lower surface: 137A with venation of 145C.
   Foliage on flowering stem.—Shape: Ovate. Apex: Acute. Base: Attenuate. Margin: Pinnatified. Venation pattern: Pinnate. Length: 9.4 cm. Width: 6.0 cm. Texture: Villous. Color of upper surface of mature foliage: More yellow than 137A with venation of closest to 145B. Color of lower surface of mature foliage: Closest to 137C with venation of closest to 145C.
Flowering description:
   Time to first flower.—Approximately 5-6 weeks from planting of rooted cutting.
   Flowering habit.—Freely flowering under outdoor growing conditions with substantially continuous blooming from spring to the first frost and year round in greenhouse environment.
   Flower arrangement.—Solitary, terminal.
Inflorescence description:
   Appearance/type.—Composite, not persistent. Shape: Round, flat. Aspect: Facing upward or outward. Outer and inner florets develop acropetally on a capitulum. Fragrance: Slight, sweet.
   Quantity of inflorescences and buds per plant.—Approximately 4 inflorescences and 6 buds at any one time.
   Lastingness of inflorescence.—Approximately 11-15 days.
   Size.—Diameter: Approximately 6.3 cm. Depth: Approximately 2.1 cm.
   Peduncle.—Strength: Strong. Aspect: At an acute angle to the stem. Length: Approximately 8.6 cm. Diameter: Approximately 1.3 mm. Texture: Dense, appressed. Color: 144A.
   Bud.—Rate of opening: Generally it takes 3 days for buds to progress from first color to fully open flowers. Shape: Round disc. Diameter at first color: Approximately 2.5 cm. Depth at first color: Approximately 1.1 cm. Immature bud color: Outer florets: 145B. Inner florets: 145B with overlay at tip of 183C. At first color: N82C.
   Corolla.—Zygomorphic.
   Outer (ray) florets.—Quantity per inflorescence: Approximately 18, arranged in a single whorl. Arrangement: Slightly overlapping. Aspect: Cupped. Shape: Obovate. Quantity of petals: 5 partially united at base, 3 large upper petals and 2 smaller lower petals. Central upper petal length: 1.3 cm. Central upper petal width: 6 mm. Apex: Obtuse. Base: Attenuate and fused to form tube. Margin: Entire. Length from apex to throat: Approximately 2 cm. Width: Approximately 7.2 mm. Lateral upper petals: length: 8 mm. Width: 6 mm. Apex: Flat to slightly obtuse. Lower petal length: 3 mm. Lower petal width: 4 mm. Texture of upper surface all petals: Glabrous. Texture of lower surface all petals: Puberulent. Color of upper surface of young and mature outer florets ray florets: N87C. Color of lower surface of mature ray florets: N87D.
   Inner (disc) florets.—Quantity: Approximately 66. Shape: Obovate, with 5 petals, 3 larger upper petals and 2 smaller lower petals. Petal apex: Obtuse. Petal base: Attenuate and fused to form tube. Length from apex of central upper petal to base of tube: 1.3 cm. Central upper lobe length: 5 mm. Central upper lobe width: 2 mm. Lateral upper lobe length: 3 mm. Lateral upper lobe width: 1.6 mm. Lower lobe length: 3 mm. Lower lobe width: 2 mm. Texture of upper surface: Glabrous. Texture of lower surface: Puberulent. Color of upper surface of petals: N87D. Color of lower surface of petals: Lighter than N87D. Tube color: Closest to N155B.
   Disc.—Diameter: Approximately 3 cm. Depth: Approximately 1.6 cm.
   Involucral bracts.—Quantity per inflorescence: Approximately 11. Arrangement: Non-imbricate, arranged in a single row. Shape: Filliform. Apex: Acute. Base: Truncate. Margin: Entire. Length: Approximately 8 mm. Width: Approximately 1 mm. Texture: Pubescent. Color at base: 154B. Color at apex: 187A.
   Peduncle.—Strength: Strong. Aspect: At an acute angle to the stem. Length: Approximately 8.6 cm. Diameter: Approximately 1.3 mm. Texture: Dense, apressed. Color: 144A.
   Reproductive organs.—Androecium: Present on disc florets only. Stamen quantity: 4, partially fused to tube. Length of free portion of filament: 2 mm. Color of filament: N155D. Anther shape: Rod. Anther length: Approximately 1.5 mm. Anther color: 161A. Amount of pollen: Moderate. Pollen color: N155B. Gynoecium: Present on ray and disc florets. There is one pistil per floret. Pistil length: 1.9 mc. Stigma shape: Funnel. Stigma length: 0.03 mm. Stigma color: N155C. Style length: 1.7 cm. Style color: Lighter than N82D at distil end, N155D at proximal end. Ovary diameter: 0.7 mm. Ovary color: 144C.
Seed and fruit production: Neither seed nor fruit production has been observed.
Disease and pest resistance: Resistance to pathogens and pests common to Scabiosa has not been observed.

What is claimed is:

1. A new and distinct cultivar of Scabiosa plant named ‘Balharbu’, substantially as herein shown and described.
